(19) United States
(12) Patent Application Publication
(10) Pub. No.: US 2010/0099158 A1
(43) Pub. Date: Apr. 22, 2010

(54) Bioenergy Production Apparatus and Method

(76) Inventor: Bruce Edgerton, Wanniassa (AU)

Correspondence Address: WOOD, HERRON & EVANS, LLP, 2700 CAREW TOWER, 441 VINE STREET, CINCINNATI, OH, 45202, US

(21) Appl. No.: 12/579,550

(22) Filed: Oct. 15, 2009

(30) Foreign Application Priority Data
Oct. 17, 2008 (AU) 2008905413

(51) Int. Cl. C12P 3/00; C12M 1/107; C12P 1/04

(52) U.S. Cl. 435/168; 435/300.1; 435/170

(57) ABSTRACT

A bioenergy production apparatus (10), including an initial digester (12) for at least partial digestion of digestible material placed therein; a collector (14) formed at or near a base of the initial digester (12), for collecting a leachate product of the digestion of the digestible material; a secondary digester (16) operatively connected to the collector (14), to at least partially digest said leachate product; and a burner (18), operatively connected to the initial digester (12) for receiving biogas output therefrom, the burner (18) being adapted to combust the biogas, and a heat exchanger (72) for transferring heat from the combustion of the biogas in the burner (18) to a fluid for use in heating the secondary digester (16), for a boiler and/or for another energy conversion process.

### CROSS-REFERENCE TO RELATED APPLICATION

This application is related to Australian Patent Application No. 2008905413, the contents of which are incorporated herein in their entirety by reference.

### FIELD OF THE INVENTION

The present disclosure relates to a bioenergy production apparatus and method. The bioenergy production apparatus and method have been developed primarily for use in processing cattle feedlot by-products. However, it will be appreciated that the apparatus and method may also be applied to processing other solid organic materials, including, for example, by-products from poultry stalls, crop residues, and the like.

### BACKGROUND

Current attempts at the production of bioenergy, particularly from the by-products of feedlots (i.e. manure) in which cattle are held for extended durations, can be very slow and inefficient. This may cause a reduction in the percentage of organic matter produced by the feedlot that is available for processes such as methanogenesis.

In addition, the high nutrient content of liquid runoff from the organic matter may have a detrimental effect on land, unless applied thereto in small volumes. Accordingly, excess manure and/or liquid runoff may need to be transported, potentially at a considerable expense, in order to achieve a sustainable nutrient balance in the surrounding crop and pasture systems.

Furthermore, due to the volume of water required to facilitate digestion, some digestion processes require over-sized digesters the effluent from which may need to be dewatered before sale. Construction and operation of such digesters can be excessively expensive and place a significant burden on water feed systems.

It should be noted that the by-products from feedlots tends to be relatively dry, having a total solids content well above 40%, and to be contaminated with dirt and other debris. Prior art systems and processes are not well adapted to process feedlot by-products, or other high-solids content or “dirty” materials, since prior art systems and processes are designed for use with relatively more moist and homogenous inputs, typically having a total solids content of much less than 40%.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY OF THE INVENTION

In a first aspect, there is provided a bioenergy production apparatus, comprising:

an initial digester for at least partial digestion of digestible material placed therein;

a collector formed at or near a base of the initial digester, for collecting a leachate product of the digestion of the digestible material;

a secondary digester operatively connected to the collector, to at least partially digest said leachate product; and

a burner, operatively connected to the initial digester for receiving biogas output from the initial digester, the burner being adapted to combust the biogas,

and a heat exchanger for transferring heat from the combustion of the biogas in the burner to the secondary digester and/or to one or more energy conversion devices.

The burner is preferably a porous burner.

The initial digester may be covered by a flexible cover, which flexible cover may be held in place by weights. The flexible cover may be used to exclude air and retain heat and thereby facilitate anaerobic digestion within the initial digester.

The initial digester may comprise a fluid inlet for moistening contents added to the initial digester. A spray device may be connected to the liquid inlet. The spray device may be fed by storm water effluent.

The initial digester may comprise a primary gas extraction line for extracting biogas from the initial digester to the burner to generate hot water. The bioenergy production apparatus may comprise a secondary gas extraction line for conveying biogas from the secondary digester to one or more energy conversion devices. The energy conversion device(s) may be the burner operatively connected to the initial digester, another burner, a boiler for steam flaking, or some other form of energy conversion device.

Biogas from the initial digester may comprise a mixture of air and highly variable levels of CH₄. This mixture may be burnt in a porous burner. Conversely, biogas from the secondary digester should be of a fairly high and consistent quality and therefore suitable for use in a boiler or other energy conversion device.

A leachate recycling line may extend between the secondary digester and the initial digester for returning leachate product output from the secondary digester to the initial digester.

The secondary digester may comprise a treated leachate output line to convey leachate product output from the secondary digester for storage.

The heat exchanger may comprise a jacket at least partially surrounding the burner to extract heat from the burner for use in an energy conversion device. The jacket may be adapted to extract heat from the burner for preheating liquid fed to the secondary digester. The jacket may be adapted to extract heat from the burner for preheating water fed to a boiler to make steam for a steam-flaking process.

The bioenergy production apparatus may comprise a plurality of initial digesters arranged to operate in parallel to feed the secondary digester.

In a second aspect, there is provided a method for the production of bioenergy, comprising:

placing digestible material in an initial digester for at least partial digestion of the digestible material to produce biogas and a leachate product;

collecting the leachate product from the initial digester;

conveying the leachate product to a secondary digester to at least partially digest the leachate product and produce biogas;

feeding the biogas from the initial digester to a burner and combusting the biogas in the burner;

feeding the biogas from the secondary digester to an energy conversion device; and

using the heat from the combustion of the biogas in the burner as a catalyst for another process.

The burner may be a porous burner.

The method may comprise the step of using the heat from the combustion of the biogas in the burner to heat the secondary digester. The method may comprise the step of using the heat from the combustion of the biogas in the burner to facilitate a steam flaking process.

The energy conversion device may be the burner associated with the initial digester, another burner, a boiler for steam flaking, or some other form of energy conversion device. Where the energy conversion device is a boiler, the method may comprise the step of using the heat from the combustion of the biogas in the burner to heat water fed to the boiler.

The method may further comprise the step of placing bulking material in the initial digester. The bulking material may form a draining bed and comprises crop residue and/or wood chips.

The method may comprise the steps of covering the initial digester with a flexible cover, and anchoring the flexible cover with moveable weights. Feedlot storm water effluent may be fed to the digestible material.

The method may comprise the step of ceasing to collect the leachate product when the bioenergy produced per unit volume of digestible material placed in the initial digester is at or below a threshold value.

The digestible material may be agitated or turned within 24 hours of completion of the collection step. After the collection step is completed, the digestible material may be agitated or turned a plurality of times over the following one to two months.

Leachate product output from the secondary digester may be returned to the initial digester to increase the moisture content of the material in the initial digester. Returning leachate product output from the secondary digester to the initial digester may also inoculate the material in the initial digester with microbes to aid digestion. Excess biomass or solids from the secondary digester may be returned to the initial digester.

The digestible material may be mechanically processed, such as by crushing and/or bulking, prior to being fed into the initial digester.

At least some of the leachate product from the initial digester may be treated to crystallise struvite therefrom. The struvite (Mg.NH₄.PO₄.6H₂O) may be collected for storage and/or sale. The treatment may comprise adding magnesium hydroxide to the leachate product from the initial digester. The struvite crystallisation may also improve the pH environment in the secondary digester and reduce the level of NH₄⁺ below concentrations that inhibit methanogenisis. A phosphate salt may also be added to facilitate removal of NH₄.

In a third aspect, there is provided a method of operating a bioenergy production apparatus according to the first aspect or a method for the production of bioenergy, said operating method comprising:

monitoring the amount of energy produced by the bioenergy production apparatus or bioenergy production method; and

claiming a benefit for the amount of energy produced.

In a fourth aspect, there is provided a method of operating a bioenergy production apparatus according to the first aspect or a method for the production of bioenergy, said operating method comprising:

monitoring the amount of digestible material processed by the bioenergy production apparatus or bioenergy production method;

calculating a reduction in carbon emissions resulting from the processing of the digestible material; and

claiming a benefit for the reduced carbon emissions.

Part of the initial digester may be formed by a ground surface of a hollow.

The initial digester may be formed from an earthen vessel.

At least part of the digestible material may be feedlot manure.

### BRIEF DESCRIPTION OF THE DRAWINGS

An embodiment will now be described, by way of non-limiting example only, with reference to the accompanying drawings, in which:

FIG. 1 is a schematic view of an embodiment of a bioenergy production apparatus in accordance with the present disclosure;

FIG. 2 is a cross-sectional view of an embodiment of an initial digester in accordance with the present disclosure;

FIG. 3 is a cross-sectional view of an embodiment of a secondary digester in accordance with the present disclosure;

FIG. 4 is a cross-sectional view of an embodiment of a bioenergy production plant in the form of a porous burner;

FIG. 5 is a schematic view of an embodiment of a bioenergy production apparatus including a plurality of initial digesters, and a boiler; and

FIG. 6 is a flow chart illustrating an embodiment of a method for the production of bioenergy in accordance with the present disclosure.

### DETAILED DESCRIPTION

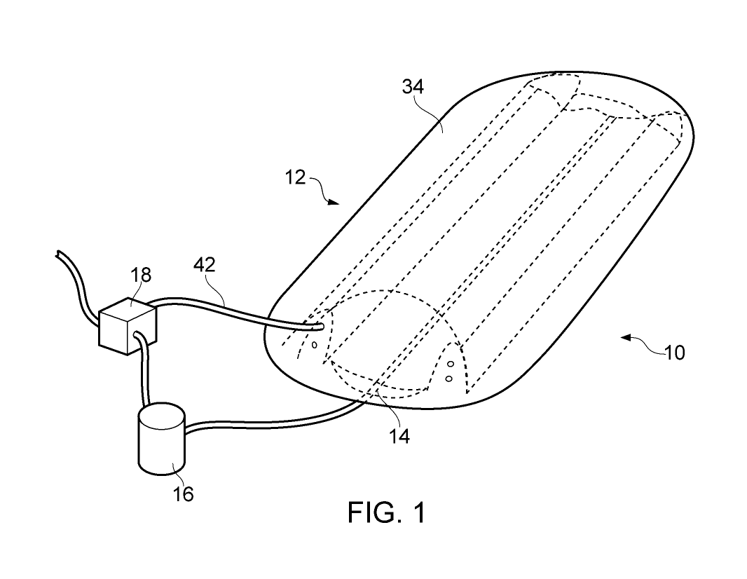

A bioenergy production apparatus 10, as shown in FIG. 1, includes at least one initial digester 12, for at least partial digestion of digestible material placed therein, a collector 14 formed in the region of the base of the at least one initial digester 12, for collecting a liquid product (leachate) of the digestion of the digestible material, and at least one secondary digester 16 operatively coupled with the collector 14 to at least partially digest the leachate product. The secondary digester 16 is a high rate digester configured to digest material therein at a higher rate than the initial digester 12. At least one bioenergy production plant, in the form of a porous burner 18, is operatively coupled to the initial digester 12 in order to receive a biogas output thereof, wherein a process occurring at the plant 18 is used for the production of bioenergy.

Corresponding parts and parts with corresponding functionality will hereinafter be referred to with corresponding reference numerals.

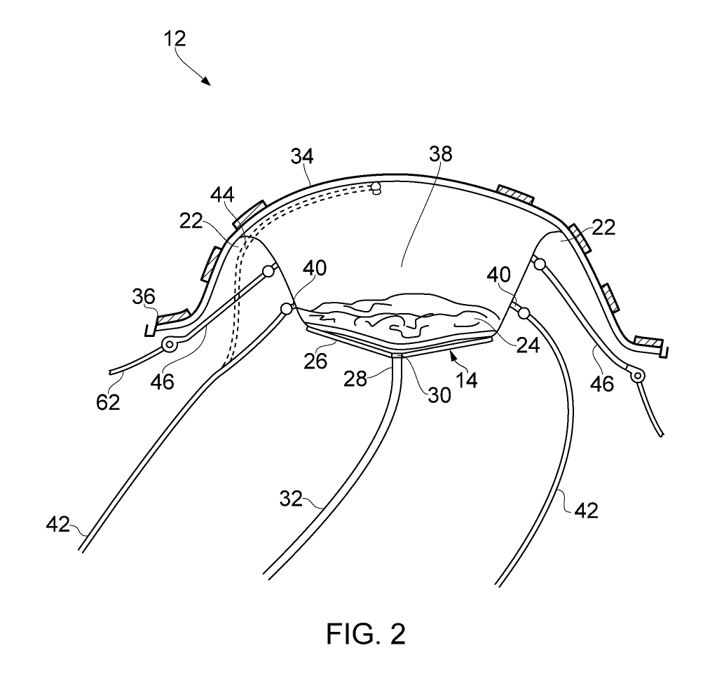

The initial digester 12, as shown in FIG. 2, may be formed by either excavating a hollow trough in the earth or, as in the present case, by constructing a silage bin including earthen berms 22 between which digestible material 24, such as manure or crop residue, is to be deposited.

At the base of the silage bin 12 is the collector 14. The collector 14 includes a sloped pan 26 of compacted earth that slopes towards an outlet 28 that may contain a filter 30, which filter would generally be adapted to filter particles greater than approximately 1 mm, or consistent with the capacity of a submersible pump located adjacent the collector. The compacted earth forming the sloped pan 26 is intended to prevent leakage of leachate (generally liquid and suspended solid runoff, with a high nutrient content, generated during digestion of the digestible material 24) into the soil beneath the silage bin 12 and the slope is designed to encourage the flow of, in particular, liquid runoff towards the outlet 28. Preferably, the pan 26 ends at the base of the respective berms 22 although it may be appropriate to end the pan 26 at some other point between the berms 22.

As the runoff is generated through digestion of the digestible material 24, it runs into the outlet 28 and is pumped via a pipeline 32 (other forms of liquid conveyance may also be used) to the secondary digester 16. The pipeline 32 (and pan 26) can be formed from any suitable material, such as PVC. Alternatively, the base of the collector 14 may be elevated above the point of entry to the secondary digester 16 (i.e. on a hill) thereby allowing runoff to flow toward the secondary digester 16 under gravity, instead of using pumps.

Some of the leachate product from the initial digester 12 may diverted from being fed into the secondary digester 16 and may be subjected to treated with magnesium hydroxide Mg(OH)₂ to crystallise struvite (Mg.NH₄.PO₄.6H₂O) therefrom. The struvite may be collected for storage and/or sale. The struvite crystallisation also improves the pH environment in the downstream secondary digester 16 and reduces the level of NH₄⁺ below concentrations that inhibit methanogenisis. A phosphate salt may also be added to facilitate removal of NH₄⁺. The high pH, low nutrient liquid remaining in the secondary digester 16 would then be digested.

The silage bin 12 also includes a flexible cover 34 made from a plastic film, such as high-density polyethylene film, (although covers of different materials and types may be suitable for other circumstances). The flexible cover 34 is weighed down by removable weights 36. These weights 36 may be in the form of used tyres due to their availability and longevity. The flexible cover 34 is intended to exclude O₂, and to trap and retain biogas within a volume 38. An advantage in using flexible covers made from plastics is that they can be rolled back while the partially digested material is removed and fresh material is added. Black plastic covers have the additional advantage of capturing solar radiation, which helps to heat the digesting material.

The silage bin 12 also includes gas extraction lines 40, 44. The gas extraction lines have inlets located above the digestible material and are used to extract biogas from the volume above the digestible material 24. Depending on the volume of digestible material 24, the time allowed for digestion, the moisture and other factors, there can be significant amounts of methane (produced by methanogenesis) and other gases trapped under the flexible cover 34. The gas extraction lines 40 convey this biogas, via a pipe 42, to a bioenergy production plant, which is discussed later. If the gas extraction lines 40 were not used, the gases would otherwise be vented into the atmosphere, particularly when the silage bin 12 is reopened in order to remove the at least partially digested digestible material. The initial digester 12, while operating, is kept at negative pressure using a blower or similar device to ensure that the biogas does not leak to the atmosphere.

The silage bin 12 also includes spray feed lines 46. The spray feed lines 46 provide moisture to the digestible material 24, which moisture can assist, in particular, anaerobic digestion. Water is primarily provided by recycling liquid from the secondary digester 16, as discussed later. Additional moisture may be desirable and can be added using stormwater harvested from feedlot runoff—this liquid runoff can also contain digestible organic matter which may further increase biogas yields.

When filling the silage bin 12, the cover 34 is usually not in place, thereby allowing machinery to deposit material in the silage bin 12 without being obstructed. Initially, a bed of bulking material of a free draining nature, such as wood chips or crop residue, is laid to elevate the digestible material 24 from the base slightly, in order to allow runoff to flow to the outlet 28 as the digestible material 24 is digested.

The digestible material 24 (generally after appropriate size reduction, such as being broken up by a tractor wheel) is then placed on top of the bulking material. Preferably, both the bulking material and digestible material are distributed evenly throughout the silage bin 12.

The flexible cover 34 is then put in place and the weights 36 added to secure the cover, and to seal the volume 38 to retain heat, moisture and gases produced during digestion of the digestible material 24. The spray feed lines 46 and gas extraction lines 40, 44 may be activated at any stage, although it is good practice to activate the spray feed lines 46 when faster digestion is desired, and the gas extraction lines 40, 44 once the digestion process has been underway for a period, thereby allowing the production of a reasonable volume of biogas before extraction is attempted. At least in the initial stages, the extraction of biogas may maintain the level of oxygen under the flexible cover 34, thereby promoting this aerobic activity. However, the oxygen will quickly deplete and digesting will become anaerobic.

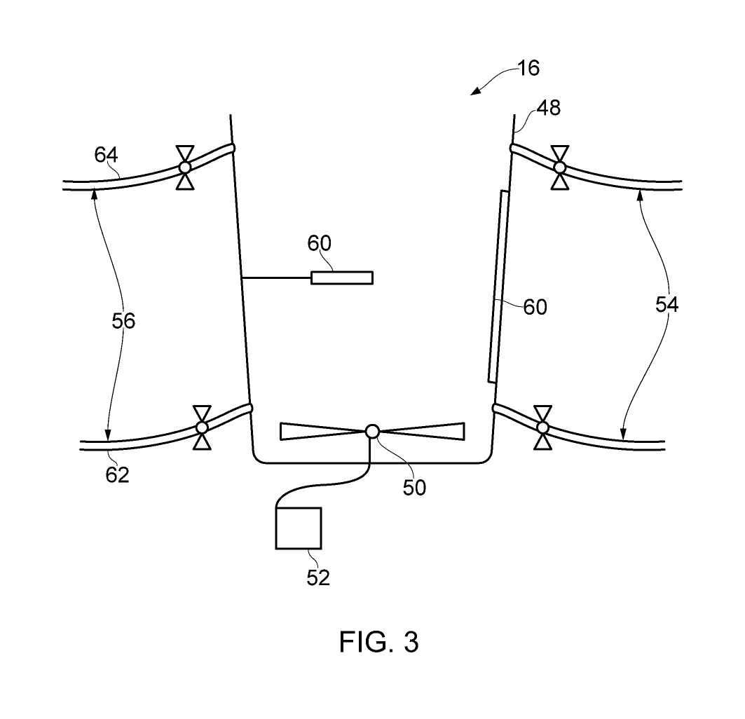

The secondary digester 16, as shown in FIG. 3, may be a continuous stirred tank reactor, or continuous-feed stirred tank reactor, (CSTR), an upflow anaerobic sludge blanket (UASB), a fixed film digester, or any other type of high rate digester. The secondary digester 16 includes a tank 48, for receiving and storing reagents for digestion, preferably an agitator 50 to ensure reagents are adequately mixed and undigested material is exposed to bacteria, a power source 52 and inlets 54 and outlets 56.

The secondary digester 16 receives one or more generally liquid inputs through the inlets 54. The inlets 54 include leachate from the silage bin 12, additional water and chemicals. The reagents may be mixed in the tank 48 or prior to being passed into the tank 48. As discussed above, treatment of a portion of the leachate in a struvite crystaliser is used to increase the pH of influent to the secondary digester 16, to pH 7-8, and to reduce the ammonia concentration below levels that inhibit methanogenisis. In some embodiments, it may be unnecessary to include the water and/or chemicals in the reaction, depending on the nature of the leachate and the percentage of ammonia therein.

To facilitate and maintain a high rate of anaerobic digestion within the tank 48, the secondary digester 16 may be heated using many methods such as a heating element 60 inside the tank, or by heating one or more of the reagents.

The outputs from the initial digester 12 may include a predominantly liquid treated effluent (liquid effluent), a predominantly solid treated effluent (solid effluent) and biogas. As perfect digestion (i.e. all material has been completely digested) cannot be obtained through this method, after biogas yields have fallen below a predetermined threshold level, digestion is considered complete. At this stage the flexible cover 34 is removed from the initial digester 12 and the spent digestible material is then removed and windrowed.

Liquid effluent (i.e. leachate) from the secondary digester 16 may be recycled periodically via a pipeline 62 to the spray feed lines 46. This reduces the amount of clean water that might otherwise be needed and helps to inoculate the initial digester 12 with microbes to aid digestion. A portion of the liquid effluent can be removed for land application to ensure salts, nutrients or metals do not accumulate to levels that inhibit methanogenisis in the apparatus 10.

Effluent with higher solid content is generally removed either periodically or once a batch has completed (i.e. when the silage bin 12 digestion process has reached an acceptable level to be deemed to have completed). The higher solid content effluent is then placed on a storage area with the spent digestible material. These solids are usually mechanically turned, to oxygenate the solids and thereby halt the anaerobic activity, preferably within the first 24 hours after completion of a batch to prevent further greenhouse gas emissions. Further turning may be required over the succeeding months in order to cure the product before sale or land application.

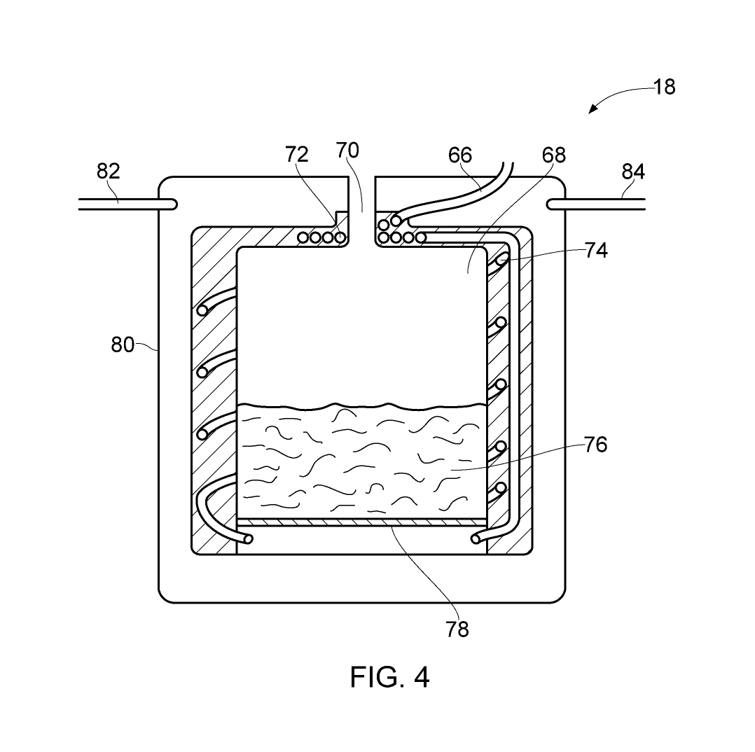

The biogas extracted from the initial digester 12 is sent via a gas outlet 64 to a bioenergy production plant in the form of porous burner 18, as shown in FIG. 4. The bioenergy production plant includes an inlet 66, a combustion chamber 68, into which chamber the biogas from the inlet 66 is delivered in order for it to undergo combustion, and an exhaust 70, for removing combusted material.

The porous burner 18 further includes a heat exchanger 72 and heat recuperator tubes 74, for pre-heating biogas from the initial digester 12 prior to combustion, a porous bed 76 and plate 78.

Biogas from the initial digester 12 (generally pre-mixed with air by valving/carburettors or any other suitable device) is fed through the inlet 66 into the heat exchanger 72. The heat exchanger 72 includes a series of spirals (which are preferably metal or ceramic, though they may be another type of suitable material) which reside above the combustion chamber 68. The spirals are heated by exhaust gas and radiant heat from the porous bed 76. The biogas passes down a series of circumferential heat recuperator tubes 74, towards the base of the burner 18, where it passes into the porous bed 76 from underneath plate 78, which is formed of porous material with lower permeability than that of the porous bed 76. Both the porous bed 76 and plate 78 are preferably made from ceramics with high Alumina content due to the high usage temperature and chemical stability thereof, although silicon carbide and other materials may also be used.

The purpose of the heat exchanger 72 and tubes 74 is to pre-heat the biogas before combustion, and the plate 78 helps to uniformly distribute the biogas throughout the bed 78 of porous material, thereby providing a better opportunity to stabilise the flame as biogas concentration and flow rate fluctuates.

The biogas passes through the plate 78 and into the porous bed 76. As the biogas passes through the matrix of the porous bed 76, biogas combustion occurs within cavities in the porous bed 76. The porous bed 76 has a high heat capacity and consequently produces higher flame speeds and extends the flammability limits of gases. As heat is transferred away from the site of combustion and into other areas of the porous bed 76, it ensures that combustion processes are stabilised, accommodating changes in biogas flow rates and concentrations (i.e. heat will approach the plate 78 as the concentration or amount of combustible biogas decreases, but as flow rates increase there is the capacity to move combustion away from the plate 78 and further up into the combustion chamber 68). In addition, the transfer of heat from the site of combustion reduces flame temperature, which in turn inhibits the formation of nitrogen oxides and the emission of carbon monoxide and unburnt hydrocarbons.

As the biogas is combusted and the exhaust gases pass through the combustion chamber 68 towards the exhaust 70, heat is radiated to the heat recuperator tubes 74 and heat exchanger 72. In addition, the burner 18 includes a heat-exchanging jacket 80. The heat exchanging jacket 80 receives radiant heat from the exhaust gas, porous bed 76 and heat recuperator tubes 74 in order to heat a fluid, such as water, to support other processes. Accordingly, the jacket 80 is also provided with an inlet 82 and outlet 84 (inflow and outflow respectively) for the fluid it is to heat.

For example, water heated within the jacket can be utilised in steam flaking plants, such as exist on many large cattle farms and can require hundreds of thousands of dollars in fuel annually, in order to remain operational. The steam flaking process improves the ability for livestock to metabolise grains and feed by breaking down grain and seed casings to make them more susceptible to digestion. This involves the steaming of the grains and feed, which requires the heating of significant amounts of water and the burning of vast quantities of fuel.

Water passing through the heat exchanging jacket 80 will quickly be heated and can produce steam. Alternatively, the water may simply be heated before being turned into steam in a boiler, which may be fed with biogas from digester 16. In either case, the steam can be utilised in a steam flaking process and provide a consequent reduction in the use of external sources of fuel, such as propane or diesel.

After steam has been used in the steam flaking process, condensate may be returned to the secondary digester 16. In addition, the jacket 80 may also, or instead, perform a heat exchanging process in pre-heating water for the digestion processes directly. In larger operations, where multiple initial digesters 12 (potentially of various different types) may be run in parallel, with one or more secondary digesters 16, it may be possible to dedicate a single bioenergy production plant 18 to pre-heating water for digestion, while other bioenergy production plants 18 help drive other processes, such as the aforementioned steam flaking process. The steam may also be used to drive a turbine or similar device, and thereby convert the stored chemical energy of the gas into mechanical energy, or electrical energy for subsequent use.

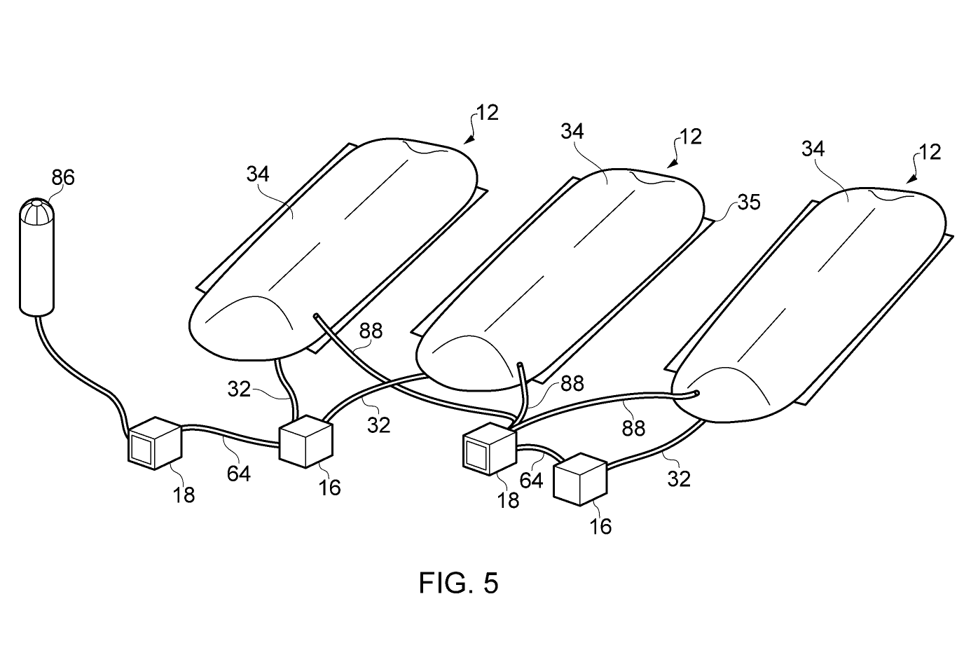

Some alternative embodiments are shown in FIG. 5, wherein a plurality of initial digesters 12 are run in parallel, with a plurality of secondary digesters 16, bioenergy production plants 18 and eventual outputs (which may be in the form of a boiler 86 of a steam flaking plant). Preheated water may be supplied from the porous burner 18 to the initial digester 12 by conduit 88 which is in fluid connection with the spray feed line 46. The number of inlets and outlets is varied between each part of the system in order to illustrate the customisable nature of the apparatus.

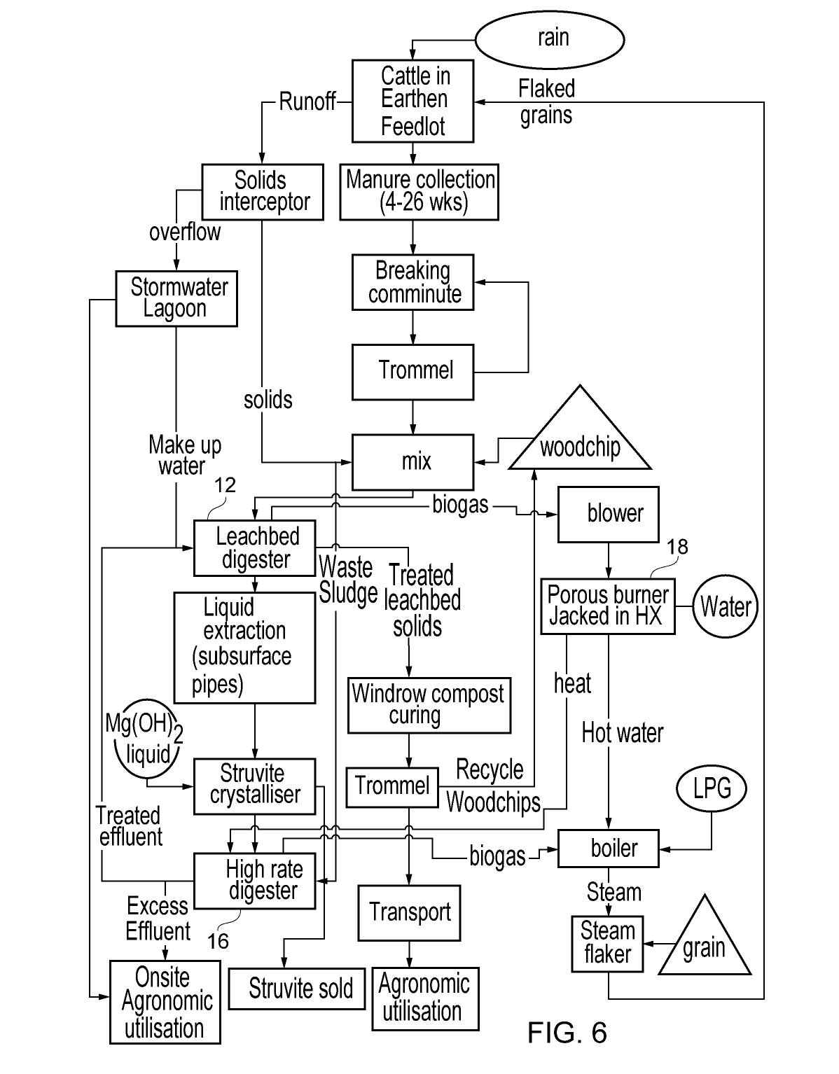

FIG. 6 shows a flowchart of a method for the production of bioenergy using the bioenergy production apparatus.

The amount of energy produced by the bioenergy production apparatus 10 is monitored to allow benefits for the amount of energy produced, such as carbon offsets, to be claimed. Where the bioenergy production apparatus 10 is used to generate electricity, a benefit can be claimed by selling the electricity produced. The amount of digestible material processed by the bioenergy production apparatus 10 is also monitored and a reduction in carbon emissions resulting from the processing of the digestible material is calculated. Again, a benefit may be claimed for the reduced carbon emissions.

It will be appreciated that the above described and illustrated apparatus 10 and method are well suited for processing relatively dry digestible materials, such as the by-products of cattle feedlots. As the initial digester 12 is able to be constructed at relatively low cost, due to using earthen walls, an earthen base and plastic film cover 34, it is economically feasible for the apparatus 10 to process the input digestible material over relatively longer timeframes than are typically required for relatively higher cost prior art systems.

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

1. A bioenergy production apparatus, comprising:
an initial digester for at least partial digestion of digestible material placed therein;
a collector formed at or near a base of the initial digester, for collecting a leachate product of the digestion of the digestible material;
a secondary digester operatively connected to the collector, to at least partially digest said leachate product;
a burner, operatively connected to the initial digester for receiving biogas output from the initial digester, the burner being adapted to combust the biogas; and
a heat exchanger for transferring heat from the combustion of the biogas in the burner to the secondary digester and/or to one or more energy conversion devices.

2. The bioenergy production apparatus as claimed in claim 1, wherein the initial digester is covered by a flexible cover, which flexible cover is held in place by weights.

3. The bioenergy production apparatus as claimed in claim 2, wherein the flexible cover is used to exclude air and retain heat and thereby facilitate anaerobic digestion within the initial digester.

4. The bioenergy production apparatus as claimed in claim 1, wherein the initial digester comprises a fluid inlet for moistening contents added to the initial digester.

5. The bioenergy production apparatus as claimed in claim 1, wherein the initial digester comprises a primary gas extraction line for extracting gas from the initial digester and keeping the initial digester at negative pressure to reduce escape of biogas around a perimeter of the flexible cover to the atmosphere.

6. The bioenergy production apparatus as claimed in claim 1, further comprising a secondary gas extraction line for conveying biogas from the secondary digester to one or more energy conversion devices.

7. The bioenergy production apparatus as claimed in claim 1, further comprising a leachate recycling line extending between the secondary digester and the initial digester for returning leachate product output from the secondary digester to the initial digester.

8. The bioenergy production apparatus as claimed in claim 1, wherein the heat exchanger comprises a jacket at least partially surrounding the burner to extract heat from the burner for use in an energy conversion device.

9. The bioenergy production apparatus as claimed in claim 1, further comprising a plurality of the initial digesters arranged to operate in parallel to feed the secondary digester.

10. A method for the production of bioenergy, comprising:
placing digestible material in an initial digester for at least partial digestion of the digestible material to produce biogas and a leachate product;
collecting the leachate product from the initial digester;
conveying the leachate product to a secondary digester to at least partially digest the leachate product and produce biogas;
feeding the biogas from the initial digester to a burner and combusting the biogas in the burner;
feeding the biogas from the secondary digester to an energy conversion device; and
using the heat from the combustion of the biogas in the burner as a catalyst for another process.

11. The method as claimed in claim 10, further comprising using the heat from the combustion of the biogas in the burner to heat the secondary digester.

12. The method as claimed in claim 10, comprising the step of using the heat from the combustion of the biogas in the burner to facilitate a steam flaking process.

13. The method as claimed in claim 10, further comprising covering the initial digester with a flexible cover, and anchoring the flexible cover with moveable weights.

14. The method as claimed in claim 10, further comprising moistening the digestible material in the initial digester.

15. The method as claimed in claim 10, further comprising returning leachate product output from the secondary digester to the initial digester.

16. The method as claimed in claim 10, further comprising ceasing to collect the leachate product when the bioenergy produced per unit volume of digestible material placed in the initial digester is at or below a threshold value.

17. The method as claimed in claim 10, further comprising treating at least some of the leachate product from the initial digester to extract struvite therefrom.

18. The method as claimed in claim 17, wherein the treating comprises adding magnesium hydroxide to the leachate product from the initial digester.

19. A method of operating a bioenergy production apparatus according to claim 1, said operating method comprising:
monitoring the amount of energy produced by the bioenergy production apparatus; and
claiming a benefit for the amount of energy produced.

20. A method of operating a bioenergy production apparatus according to claim 1, said operating method comprising:
monitoring the amount of digestible material processed by the bioenergy production apparatus;
calculating a reduction in carbon emissions resulting from the processing of the digestible material; and
claiming a benefit for the reduced carbon emissions.
